(19) United States
(12) Patent Application Publication
(10) Pub. No.: US 2025/0271422 A1
(43) Pub. Date: Aug. 28, 2025

(54) METHOD FOR MANUFACTURING MULTIPLE SILVER NANOGAP SHELL NANOPROBE, MULTIPLE SILVER NANOGAP SHELL NANOPROBE MANUFACTURED THEREBY, AND METHOD FOR DIAGNOSING LIQUID BIOPSY BASED ON SERS NANOPROBE USING SAME

(71) Applicant: INDUSTRY-UNIVERSITY COOPERATION FOUNDATION HANYANG UNIVERSITY ERICA CAMPUS, Ansan-si (KR)

(72) Inventors: In Jun HWANG, Ansan-si (KR); Chan Hee CHOI, Ansan-si (KR); Hong Won KIM, Ansan-si (KR); Jong Ho KIM, Ansan-si (KR)

(21) Appl. No.: 19/199,236

(22) Filed: May 5, 2025

Related U.S. Application Data
(63) Continuation of application No. PCT/KR2024/003737, filed on Mar. 26, 2024.

(30) Foreign Application Priority Data
Apr. 6, 2023 (KR) 10-2023-0045128
Apr. 20, 2023 (KR) 10-2023-0051838

(51) Int. Cl. G01N 33/543; G01N 21/65; G01N 33/531; G01N 33/574

(52) U.S. Cl. CPC G01N 33/54346; G01N 21/658; G01N 33/531; G01N 33/54326; G01N 33/57438; G01N 2470/04

(57) ABSTRACT

Provided is a method for preparing a multiple silver nanogap shell nanoprobe, comprising preparing Mesoporous Silica Nanoparticles (MSN); and mixing the mesoporous silica nanoparticles with a silver precursor and a Raman marker material to grow silver from the silver precursor on the mesoporous silica nanoparticles. Provided is a liquid biopsy diagnostic method using the multiple silver nanogap shell nanoprobe.

### CROSS-REFERENCE TO RELATED APPLICATION

This application is a bypass continuation of pending PCT International Application No. PCT/KR2024/003737, which was filed on Mar. 26, 2024, and which claims priority to and the benefit of Korean Patent Application No. 10-2023-0045128, which was filed in the Korean Intellectual Property Office on Apr. 6, 2023, and Korean Patent Application No. 10-2023-0051838, which was filed in the Korean Intellectual Property Office on Apr. 20, 2023 the disclosure of which are incorporated herein by reference in its entirety.

### TECHNICAL FIELD

The present invention relates to a method for preparing a multiple silver nanogap shell nanoprobe, a multiple silver nanogap shell nanoprobe prepared thereby, and a method for diagnosing liquid biopsy based on SERS nanoprobe using the same, and more specifically, to a method for preparing a multiple silver nanogap shell nanoprobe, a multiple silver nanogap shell nanoprobe prepared thereby, and a method for diagnosing liquid biopsy based on SERS nanoprobe using the same, wherein the method is capable of preparing porous particles capable of inducing a nanogap structure and effectively diagnosing pancreatic cancer through the same.

### BACKGROUND ART

A detection technology based on Surface-enhanced Raman scattering (hereinafter, referred to as SERS) is receiving much attention as a new disease diagnosis method that may replace the existing fluorescence/absorption detection method because sensitivity is very excellent and multi-detection is possible.

However, it is important to develop a nanoprobe structure in which the high-sensitivity SERS signal is highly dependent on the structure of the SERS nanoprobe and still exhibits an ultra-high-sensitivity SERS signal, and in particular, it is necessary to develop a nanoprobe having excellent signal strength and signal stability compared to particles.

Meanwhile, pancreatic cancer diagnosis is performed through CT and MRI imaging. Image-based diagnosis is difficult to find small cancers of less than 1 cm, which is the biggest problem that increases the mortality rate of pancreatic cancer patients. Therefore, to overcome this problem, many studies have been conducted to diagnose pancreatic cancer through the detection of a liquid biopsy biomarker, but the existing fluorescence/absorption-based immunoassay method has a problem in that the detection sensitivity is not good, and thus the diagnosis accuracy of pancreatic cancer is very low.

Therefore, there is a need to develop a novel nanoprobe manufacturing method capable of effectively early diagnosing a disease such as pancreatic cancer and a technology capable of diagnosing pancreatic cancer using the nanoprobe manufactured therefrom.

### DISCLOSURE

### Technical Problem

The present disclosure is directed to provide a novel method for preparing a nanoprobe capable of effectively diagnosing a disease such as pancreatic cancer in early stage, and a diagnostic method capable of diagnosing pancreatic cancer using the nanoprobe prepared therefrom.

### Technical Solution

To achieve the above objective, the present invention provides a method for preparing a multiple silver nanogap shell nanoprobe, the method comprising the steps of preparing Mesoporous Silica Nanoparticles (MSN); and mixing the mesoporous silica nanoparticles with a silver precursor and a Raman marker material to form a silver structure having a nanogap from the silver precursor in the mesoporous silica nanoparticles.

In an embodiment of the present invention, the Raman marker material is mixed with the mesoporous silica nanoparticles before forming the silver structure completely from the silver precursor.

In an embodiment of the present invention, the mesoporous silica nanoparticles have a porous structure formed inside the Mesoporous Silica Nanoparticles (MSN), and the silver precursor penetrates the porous structure to form the silver structure in the porous structure.

In an embodiment of the present invention, the Raman marker material is confined in the nanogap of the silver structure.

The present invention also provides a multiple silver nanogap shell nanoprobe prepared by the method mentioned above.

In an embodiment of the present invention, the multiple silver nanogap shell nanoprobe comprises: a porous structure formed inside a Mesoporous Silica Nanoparticles (MSN); and a silver structure formed in the porous structure.

In an embodiment of the present invention, the silver structure in the porous structure has at least two nanogaps.

In an embodiment of the present invention, the Raman marker material is confined in the nanogap.

In an embodiment of the present invention, wherein the multiple silver nanogap shell nanoprobe is a probe for detecting Surface-Enhanced Raman Scattering (SERS).

The present invention also provides a liquid biopsy diagnostic method using the multiple silver nanogap shell nanoprobe mentioned above, comprising: preparing magnetic particles bound with an antigen; capturing a detection antibody in the multiple silver nanogap shell nanoprobe mentioned above; mixing the magnetic particles and the multiple silver nanogap shell nanoprobe to prepare an immunosandwich composite; and diagnosing the presence or absence and concentration by mapping raman scattering signal from the immunosandwich composite.

In an embodiment of the present invention, the antigen is a pancreatic cancer antigen.

In an embodiment of the present invention, the antigen is captured by the magnetic particle through a capture antibody.

The present invention also provides a liquid biopsy diagnostic probe comprising: Mesoporous Silica Nanoparticles (MSN) having a porous structure formed therein; a silver structure formed in the porous structure; and a Raman marker material, wherein the silver structure has at least two nanogaps, and the Raman marker material is confined in the nanogaps.

In an embodiment of the present invention, the liquid biopsy diagnostic probe is for a Surface-enhanced Raman scattering (SERS)-based liquid biopsy.

### Advantageous Effects

The preparation method according to the present invention has the advantages of easily controlling the nanogap structure of the nanostructure and enabling synthesis at a time within 20 minutes. In addition, the silver nanogap shell manufactured by the method according to the present invention exhibits a strong SERS signal and has very excellent signal strength and signal stability compared to existing SERS particles, and using this, CA19-9, which is one of the pancreatic cancer liquid biopsy biomarkers, can be quantitatively detected with high sensitivity.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic diagram of the synthesis of AgMPS, which is a nanoprobe according to an embodiment of the present invention, a) a schematic diagram of the synthesis of AgMPS mediated by a porous template, and b) a chemical structure of various Raman-labeled chemicals used in AgMPS.

FIG. 2 shows the results of AgMPS structural analysis, showing a) optical image and b) the UV-Vis absorption spectra of a reaction solution for the synthesis of AgMPS comprising 4-FBT as a function of time. c) TEM image of AgMPS over time. d) TEM image of AgMPS [4-FBT] obtained 600 seconds after reaction. e) Size distribution of AgMPS [4-FBT]. f) Ag nanoparticles on a single AgMPS and g) the distribution of nanogaps on AgMPS [4-FBT] over time.

FIG. 3 shows the results of SERS characterization of AgMPS, a) a SERS spectrum of AgMPS including various types of Raman label chemicals. b) SERS intensity at 1075 cm-1 of AgMPS [4-FBT] obtained at 10 sec and 3600 sec. c) SERS enhancement mapping of a single AgMPS [4-FBT] particle and d) SERS enhancement factor distribution. e) SERS intensities of AgMPS [4-FBT] and 4-FBT-Ag@MSN. f) Change in SERS intensity at 540 cm-1 after treatment of AgMPS [4-CBT] and 4-CBT-Ag@MSN with reducing agent NaBH4 to remove Raman chemicals. g) Stability analysis results of SERS intensities of AgMPS [4-FBT] and 4-FBT-Ag@MSN over time.

FIG. 4 is a schematic diagram of a) AgMPS SERS nanoprobe-based immunoassay for CA19-9 detection, as a result of detecting CA19-9, which is a pancreatic cancer antigen, using the AgMPS SERS nanoprobe prepared according to an embodiment of the present invention. b) SERS mapping image of the measured SERS nanoprobe sandwich complex dropped onto a glass array after completion of the analysis. c) Representative Raman spectra obtained from a spot of SERS mapping (yellow circle of mapping image). d) The total SERS intensity of the SERS nanoprobe sandwich complex as a function of CA19-9 concentration.

FIG. 5 shows the results of CA19-9 detection in human plasma using the SERS nanoprobe immunoassay, which is a) an SERS mapping image of the SERS nanoprobe sandwich complex measured in a glass array after incubation with plasma samples containing CA19-9 at different concentrations. b) Representative SERS mapping image of the SERS nanoprobe sandwich complex on human plasma samples after completion of analysis. c) Quantification of CA19-9 from plasma samples measured by SERS nanoprobe immunoassay. d) Coefficient of variation (CV) and recovery rate for CA19-9 detection from human plasma samples measured by SERS nanoprobe immunoassay.

### BEST MODE

Hereinafter, exemplary embodiments of the present invention will be described in detail with reference to the accompanying drawings.

Before describing the present invention in detail, the terms or words used in the present specification should not be interpreted as being unconditionally limited to typical or dictionary meanings, and the inventors of the present invention may appropriately define and use the concepts of various terms in order to describe their invention in the best way.

Furthermore, it should be noted that these terms or words should be interpreted as meanings and concepts consistent with the technical spirit of the present invention.

That is, the terms used in the present specification are only used to describe the preferred embodiments of the present invention, and are not intended to specifically limit the contents of the present invention.

It should be noted that these terms are defined in consideration of various possibilities of the present invention.

In addition, in the present specification, a singular expression may include a plural expression unless the context clearly indicates a different meaning.

In addition, it should be noted that even if it is similarly expressed in plural, it may include a singular meaning.

When a component is described as “including” another component throughout the specification, it may mean that any other component may be further included, rather than excluding any other component, unless otherwise described.

Furthermore, if a component is described as being “inside” or “connected and installed” to another component, the component may be directly connected to or in contact with the other component.

To solve the above-described problems, the present invention prepares a SERS nanoprobe by a method of growing a silver nanostructure on a porous particle (MSN), which is a template having a nanogap structure, and then introduces an antibody through EDC/NHS reaction to prepare a SERS nanoprobe.

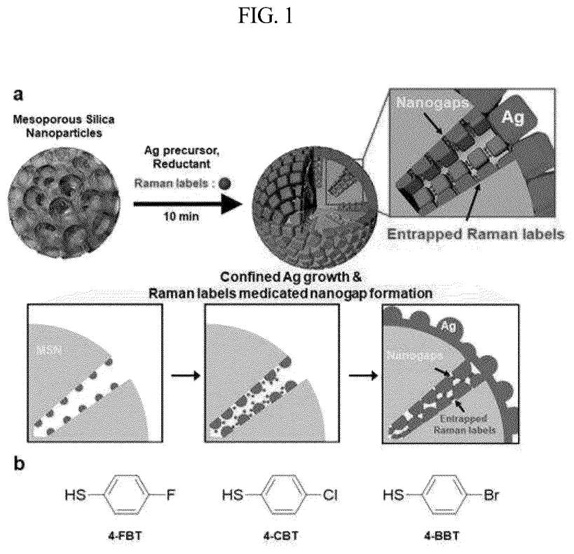

FIG. 1 is a schematic diagram of the synthesis of AgMPS, which is a nanoprobe according to an embodiment of the present invention, a) a schematic diagram of the synthesis of AgMPS mediated by a porous template, and b) a chemical structure of various Raman-labeled chemicals used in AgMPS.

Referring to FIG. 1, in the AgMPS synthesis method according to the present disclosure, Mesoporous Silica Nanoparticles (MSN) is first synthesized, and then silver structure is formed from a silver precursor in a porous structure of the mesoporous silica nanoparticles. At this time, silver (Ag) precursor penetrates the porous structure and grows to have a gap in the silver structures where Raman markers are entrapped and confined between these gaps. For such entrapment of the Raman markers between nanogap, it is important to mix Raman marker material with the silver precursor before the silver structure is completely formed from the precursor.

The nanoprobe according to the present invention has at least two nanogaps in silver structures inside a porous structure and the Raman marker is entrapped and confined in the nanogap. Hereinafter, the manufacturing method will be described in more detail through Examples.

In addition, the present invention provides a method for accurately quantitatively detecting the concentration of CA19-9 through SERS mapping, after capturing magnetic particles, to which the capture antibody is fixed, by reacting the same with a SERS antigen, forming an immunosandwich complex by treating an CA19-9 nanoprobe into which the detection antibody is introduced, and then applying the immunosandwich complex to a glass array chip.

Accordingly, the present invention provides a method for preparing a silver nanogap shell having excellent sensitivity and SERS signal stability and provides a nanoprobe which can quickly diagnose pancreatic cancer using the method and can be applied to the diagnosis of various diseases based on a liquid biopsy, and a method for utilizing the nanoprobe.

Hereinafter, the present invention will be described in more detail through Examples. However, the scope of the present disclosure is not limited by the following examples.

### EMBODIMENT

### Synthesis of Mesoporous Silica Nanoparticles (MSN)

First 0.068 g of TEA was added to 25 ml of water and stirred gently at 80° C. in an oil bath under magnetic stirring for 0.5 hours. Then, 380 mg of CTAB and 84 mg of NaSal were added to the solution and stirring was continued for 1 hour. Then, a mixture of 2 ml TEOS and 1.6 ml BTEE was added to the water-CTAB NaSal-TEA solution with gentle stirring (300 rpm) for 12 hours. The product was collected by high-speed centrifugation and washed several times with ethanol to remove residual reactant, then the collected product was extracted with HCl and methanol solution three times at 60.0° C. for 6 h, the template was removed and vacuum dried at room temperature overnight. MSN (15 mg) was then dispersed in 1 mL of ethanol containing 60 μL of MPTS and 10 μL of ammonium hydroxide. The resulting mixture was stirred at room temperature for 12 h, and the MPTS-functionalized MSN was washed several times with ethanol to remove excess reagent via centrifugation.

### Synthesis of AgMPS (Ag Mesoporous Shell)

1. 25 ml portion of ethylene glycol (EG) was placed in a 50 ml conical tube and 5 mg of the PVP portion was added to the ethylene glycol.
2. The reaction mixture was placed in a round bottom flask and the reaction mixture was stirred at 600 rpm at 250 C.
3. 25 ml of ethylene glycol was placed in a 50 ml conical tube and 17 mg of AgNO3 was added to the ethylene glycol.
4. The reaction mixture was stirred at 250 rpm. on a 3D shaker.
5. 13.3 ul of 4-FBT was added to 486.7 ul of ethanol. (4-FBT stock from 250 mM)
6. MSN treated with 3-mercaptopropyltrimethoxysilane (3-mercaptopropyltrimethoxysilane) was added to the round bottom flask to increase dispersibility before reaction.
7. 25 ml of AgNO3/EG, a well-dispersed silver precursor, was added to the round bottom flask and the reaction mixture was stirred at 25° C. to 760 rpm.
8. 16 ul of octylamine, a reducing agent for reducing the silver precursor, was added to the round bottom flask.
9. After 10 minutes, the Raman marker material, 4-FBT stock 10 ul, was added to the solution in which the silver precursor undergoing reduction (i.e., before silver structure growth complete), at which time the color of the reaction mixture was clear red.
10. After 1 h, the reaction mixture was transferred to a 50 ml conical tube and centrifuged at 10000 rpm for 5 min with ethanol to remove excess reagent (repeat 5 times) (2 or more tubes).
11. 1 ml of AgMPS dissolved in ethanol was stored in a 15 ml conical tube at 4°.

Through the above process, in the mesoporous porous structure, first, the silver precursor is adsorbed on the surface functionalized with a thiol group. Thereafter, the silver precursor is reduced by octylamine, and at this time, the silver precursor is simultaneously reduced in several places in the mesoporous porous structure, and then a Raman marker material (Raman marker material) such as 4-FBT is added, and at this time, the Raman marker material is intercalated between the silver nanoparticles to form a nanogap.

### AgMPS SERS Nanoprobe-Based Immunoassay for CA19-9 Detection

Capture antibody (CAb)-MB (8×106 25 μL per mL) was dispersed in 425 μL of a 1% BSA/PBS (0.1 m, pH 7.4) solution. The 20 μL target biomarker was added to the CAb-MB solution at a final concentration of 5 to 400 U mL-1.

The resulting mixture was incubated for 2 h with gentle shaking at room temperature. The CAb-MB capture target biomarker was washed several times with 0.1% PBS-T (0.1 m, pH 7.4) and PBS (0.1 m, pH 7.4), and then incubated with SERS NP [4-BBT] (25 μL, 0.2 mg/mL-1) (final solution volume=0.5 mL) or detection antibody (DAb)-SERS NP [4-FBT] at room temperature for 2 hours.

Finally, unbound SERS nanoprobe was removed by washing CAb-MB several times with 0.1% PBS-T (0.1 m, pH 7.4) and PBS (0.1 m, pH 7.4). To collect the SERS signal from the CAb-MB, the entire solution of CAb-MB was dropped onto the microwell substrate.

### CA19-9 Detection in Patient Plasma Samples

Patient plasma samples were provided by Seoul National University Bundang Hospital. Capture antibody (CAb)-MB (8×106, 25 μL per mL) was dispersed in 425 μL of 1% BSA/PBS (0.1 m, pH 7.4) solution. To the CAb-MB solution was added 20 μL of the target biomarker at a final concentration between 5 and 400 U mL-1. The resulting mixture was incubated in PBS for 2 hours with gentle shaking at room temperature. The CAb-MB capture target biomarker was washed several times with 0.1% PBS-T (0.1 m, pH 7.4) and PBS (0.1 m, pH 7.4), and then incubated with SERS NP [4-BBT] (25 μL, 0.2 mg/mL) or detection antibody (DAb)-SERS NP [4-FBT] or SERS NP [4-BBT] (25 μL, 0.2 mg/mL) for 2 hours at room temperature (final solution volume-0.5 mL). Finally, CAb-MB was washed several times with 0.1% PBS-T (0.1m, pH 7.4) and PBS (0.1 m, pH 7.4) to remove unbound SERS nanoprobe, the entire solution of CAb-MB was dropped on a microwell substrate to collect SERS signals from CAb-MB, and SERS mapping was performed according to a known method.

### EXPERIMENTAL EXAMPLE

### AgMPS Structure Analysis

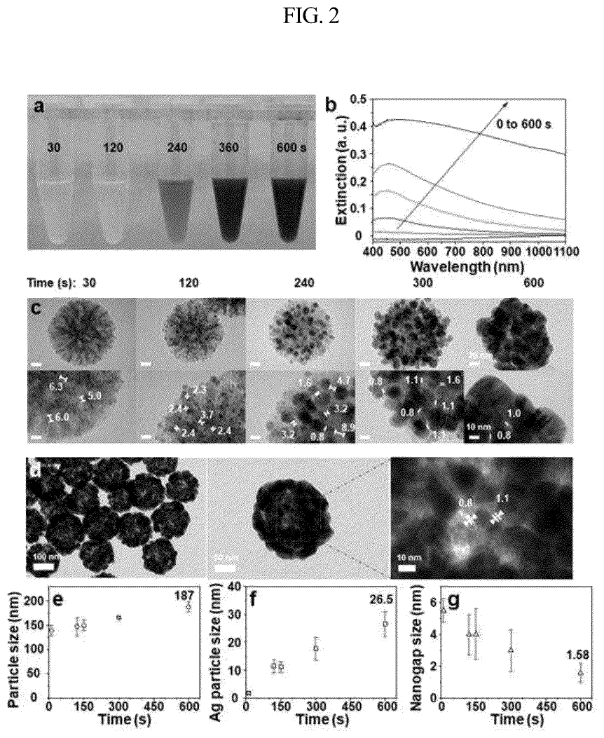

FIG. 2 shows the results of AgMPS structural analysis, showing a) optical image and b) the UV-Vis absorption spectra of a reaction solution for the synthesis of AgMPS comprising 4-FBT as a function of time. c) TEM image of AgMPS over time. d) TEM image of AgMPS [4-FBT] obtained 600 seconds after reaction. e) Size distribution of AgMPS [4-FBT]. f) Ag nanoparticles on a single AgMPS and g) the distribution of nanogaps on the AgMPS [4-FBT] over time.

Referring to FIG. 2, it can be confirmed that multiple nanogaps are formed in the mesoporous porous structure as shown in FIG. 1.

### AgMPS SERS Attribute Analysis

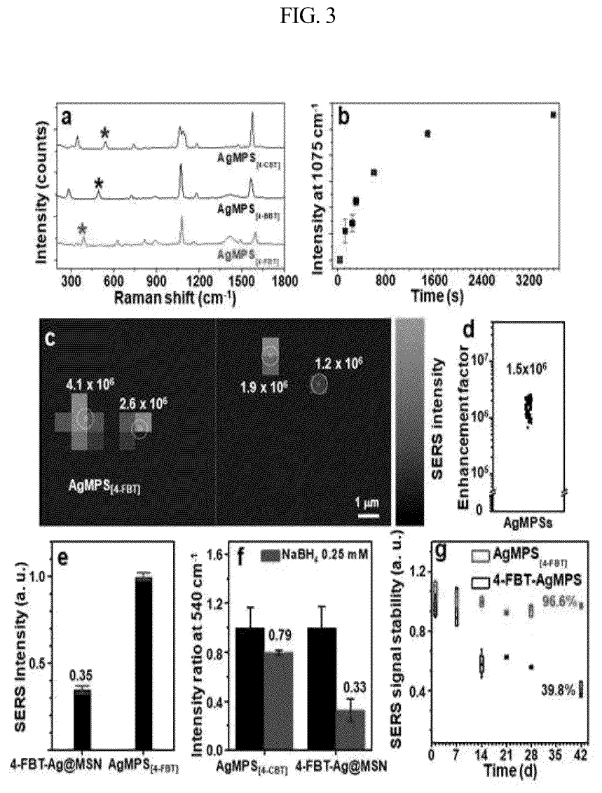

FIG. 3 shows a result of SERS characterization of AgMPS, a) a SERS spectrum of AgMPS including various types of Raman label chemicals. b) SERS intensity at 1075 cm-1 of AgMPS [4-FBT] obtained at 10 sec and 3600 sec. c) SERS enhancement mapping of a single AgMPS [4-FBT] particle and d) SERS enhancement factor distribution. e) SERS intensities of AgMPS [4-FBT] and 4-FBT-Ag@MSN. f) Change in SERS intensity at 540 cm-1 after treatment of AgMPS [4-CBT] and 4-CBT-Ag@MSN with reducing agent NaBH4 to remove Raman chemistry. g) Stability analysis results of SERS intensities of AgMPS [4-FBT] and 4-FBT-Ag@MSN over time.

More specifically, (a) represents multiple signals of various Raman marker materials, and (b) represents the Raman signal intensity according to the reaction time. Referring to this, it can be seen that the nanoprobe according to the present invention exhibits a multiple effect even in various Raman-labeled materials and increases with reaction time.

(c, d) is an analysis of the SERS enhancement factor of the synthesized multiple silver nanogap particles, and it can be seen that the Raman signal in the silver nanogap particles increases by 1.5×106.

In (e), the Raman marker material added during the synthesis process was silver multiple nanogap particles, and after the synthesis, the Raman marker material was washed by centrifugation, and post-treated was Ag@MSN. It can be seen that the nanogap is well formed when the Raman marker material is introduced into the in-situ during the synthesis process, and the SERS signal is better emitted by the well-formed nanogap. This suggests that the nanoprobe according to the present invention is formed only when the Raman marker material is added to the reaction solution during the formation of the nanogap structure.

In (f), AgMPS (multiple silver nanogap particles) and Ag@MSN were prepared using NaBH4, which is a material for reduction desorption. It can be seen that the multiple silver nanogap particles show a stable SERS signal because the Raman marker material is well intercalated in the nanogap structure, whereas in the case of Ag@MSN, the Raman marker material is not formed in the nanogap in the porous structure, but is adsorbed on the surface of Ag@MSN, and is separated during the desorption process, and then the ERS signal is significantly reduced.

In (g), the stability of the SERS signal was confirmed as in (e,f). That is, when the SERS signal is measured under the same conditions over time, Ag@MSN shows a stable SERS signal because the Raman marker material easily drops over time, whereas the multiple silver nanogap particles prepared by adding the label marker material during the nanoprobe synthesis of the label marker material according to the present invention, more precisely, during the formation of the silver structure in the porous structure, are well confined in the nanogap structure.

### CA19-9 Detection Results Using AgMPS SERS Nanoprobe-Based Immunoassay

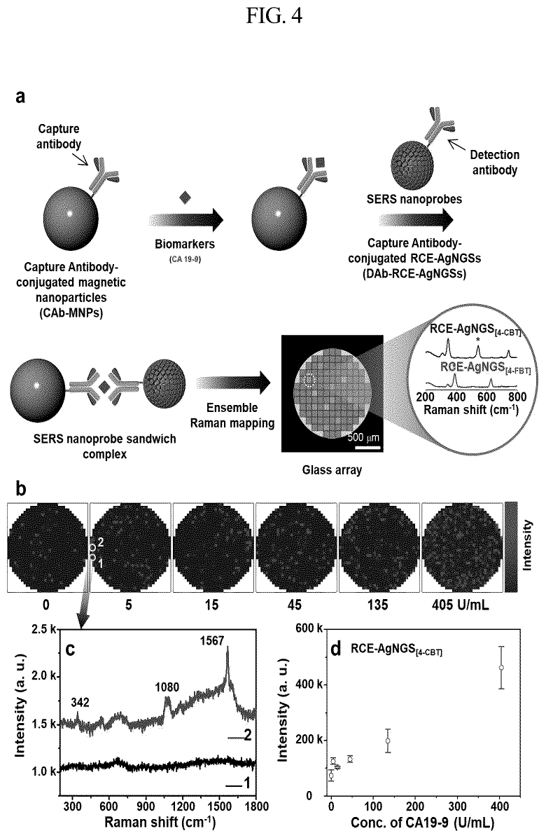

FIG. 4 is a schematic diagram of a) AgMPS SERS nanoprobe-based immunoassay for CA19-9 detection, as a result of detecting CA19-9, which is a pancreatic cancer antigen, using a AgMPS SERS nanoprobe prepared according to an embodiment of the present invention. b) SERS mapping image of the measured SERS nanoprobe sandwich complex dropped onto a glass array after completion of the analysis. c) Representative Raman spectra obtained from a spot of SERS mapping (yellow circle of mapping image). d) The total SERS intensity of the SERS nanoprobe sandwich complex as a function of CA19-9 concentration.

Referring to FIG. 4, through (a), a method of detecting CA19-9 using SERS nanoprobe (multiple silver nanogap particles with an antibody) can be confirmed, and first, in the CA19-9 capturing step, a magnetic particle (CAb-MNP) to which an antibody specifically binding to CA19-9, which is an antigen, is fixed is reacted with CA19-9. In the formation of a SERS nanoprobe sandwich complex, the SERS nanoprobe according to the present invention reacts with magnetic particles captured by CA19-9. Thereafter, the composite is dropped on a glass array capable of SERS measurement, dried, and SERS measurement is performed.

(b) is data measured by SERS according to the concentration of CA19-9, and it can be confirmed that it is concentration-dependent.

(c) shows that a characteristic SERS signal of CBT (Chlorobenzenethiol) appears during mapping, and (d) is quantified using this signal. Referring to this, it can be seen that the signal increases according to the concentration.

### CA19-9 Detection Results in Human Plasma Using SERS Nanoprobe Immunoassay

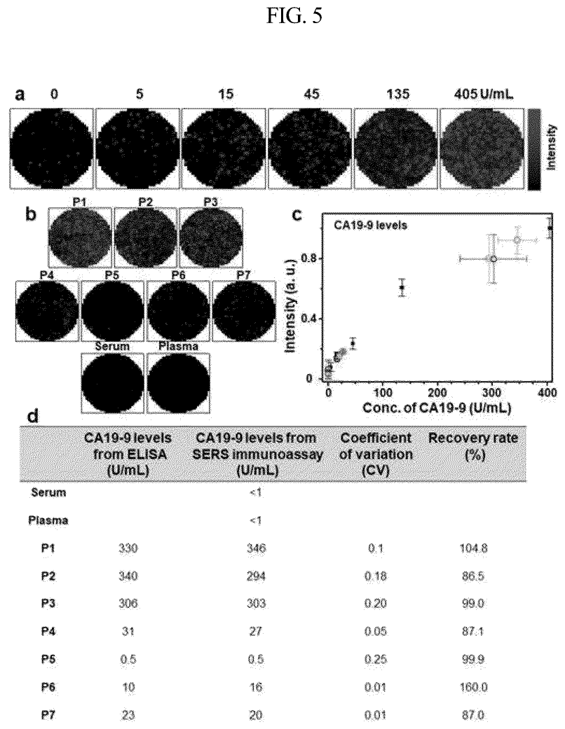

FIG. 5 shows the results of CA19-9 detection in human plasma using the SERS nanoprobe immunoassay, which is a) an SERS mapping image of the SERS nanoprobe sandwich complex measured in a glass array after incubation with plasma samples containing CA19-9 at different concentrations. b) Representative SERS mapping image of the SERS nanoprobe sandwich complex on human plasma samples after completion of analysis. c) Quantification of CA19-9 from plasma samples measured by SERS nanoprobe immunoassay. d) Coefficient of variation (CV) and recovery rate for CA19-9 detection from human plasma samples measured by SERS nanoprobe immunoassay.

Referring to FIG. 5, (a) is a mapping image like (b) of FIG. 4. (Calibration curve) (b) is data obtained by quantifying the amount of CA 19-9 in plasma samples of pancreatic cancer patients collected at Seoul National University Bundang Hospital, and (c) is a mapping image, which is quantified by plot. (d) is a table in which how effective the SERS nanoprobe-based immunoassay is, and is a table in which CV and Recovery rate are calculated. Referring to this, the SERS nanoprobe-based immunoassay according to the present invention has an LOD (minimum detection limit) of 0.025 U/mL, which is 10 times or more superior to the commercially available ELISA (ELISA LOD: 0.3 U/mL, Thermo Fisher Scientific). In addition, CV was 0.11, which is very reliable, and Recovery rate was 103%.

Therefore, the nanoprobe according to the present invention can be used as a liquid biopsy diagnostic probe, and in particular, is very effective as a liquid biopsy diagnostic probe based on Surface-enhanced Raman scattering (hereinafter, SERS).

What is claimed is:

1. A method for preparing a multiple silver nanogap shell nanoprobe, comprising preparing Mesoporous Silica Nanoparticles (MSN); and
mixing the mesoporous silica nanoparticles with a silver precursor and a Raman marker material to grow silver from the silver precursor on the mesoporous silica nanoparticles.

2. The method of claim 1, wherein the Raman marker material is mixed with the mesoporous silica nanoparticles before the silver is completely grown.

3. The method of claim 2, wherein the nanoprobe has a porous structure formed inside the Mesoporous Silica Nanoparticles (MSN), and the silver precursor penetrates the porous structure to grow silver in the porous structure.

4. The method of claim 2, wherein the Raman marker material is confined in a porous structure.

5. A multiple silver nanogap shell nanoprobe prepared according to claim 4.

6. The multiple silver nanogap shell nanoprobe according to claim 5, wherein the multiple silver nanogap shell nanoprobe comprises: the porous structure formed inside a Mesoporous Silica Nanoparticles (MSN); and
a silver structure formed in the porous structure.

7. The multiple silver nanogap shell nanoprobe according to claim 6, wherein the silver structure in the porous structure has at least two nanogaps.

8. The multiple silver nanogap shell nanoprobe according to claim 7, wherein the Raman marker material is confined in the nanogap.

9. The multiple silver nanogap shell nanoprobe according to claim 8, wherein the multiple silver nanogap shell nanoprobe is a probe for detecting Surface-Enhanced Raman Scattering (SERS).

10. A liquid biopsy diagnostic method using the multiple silver nanogap shell nanoprobe according to claim 9, comprising:
preparing magnetic particles bound with an antigen;
capturing a detection antibody in the multiple silver nanogap shell nanoprobe;
mixing the magnetic particles and the multiple silver nanogap shell nanoprobe to prepare an immunosandwich composite; and
diagnosing the presence or absence and concentration by mapping raman scattering signal from the immunosandwich composite.

11. The liquid biopsy diagnostic method according to claim 10, wherein the antigen is a pancreatic cancer antigen.

12. The liquid biopsy diagnostic method according to claim 10, wherein the antigen is captured by the magnetic particle through a capture antibody.

13. A liquid biopsy diagnostic probe comprising:
Mesoporous Silica Nanoparticles (MSN) having a porous structure formed therein;
a silver structure formed in the porous structure; and
a Raman marker material, wherein the silver structure has at least two nanogaps, and the Raman marker material is confined in the nanogaps.

14. The liquid biopsy diagnostic probe according to claim 13, wherein the liquid biopsy diagnostic probe is for a Surface-enhanced Raman scattering (SERS)-based liquid biopsy.
